(19) United States
(12) Patent Application Publication
(10) Pub. No.: US 2007/0122357 A1
(43) Pub. Date: May 31, 2007

(54) Dentifrice composition

(75) Inventor: William Michael Glandorf, Mason, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/545,266

(22) Filed: Oct. 10, 2006

Related U.S. Application Data
(60) Provisional application No. 60/740,538, filed on Nov. 29, 2005.

(51) Int. Cl. A61K 8/21

(52) U.S. Cl. 424/52

(57) ABSTRACT

Dentifrice compositions which provide chemical cleaning of the oral surfaces with a combination of surfactant, chelant, and pH; further this dentifrice composition can minimize the use of abrasives to provide a gentle cleaning of the oral surfaces.

### CROSS REFERENCE TO RELATED APPLICATIONS

This application claims the benefit of U.S. Provisional Application No. 60/740,538 filed Nov. 29, 2005.

### FIELD OF THE INVENTION

The present invention relates to dentifrice compositions that can provide a chemical cleaning of the surfaces of the oral cavity. Further, this invention relates to a method of treating or preventing oral conditions. Dentifrice compositions of the present invention are suitable for use by humans or animals.

### BACKGROUND OF THE INVENTION

A satisfactory dentifrice composition cleans and removes debris from the surfaces of the oral cavity, in particular the hard tissue surfaces such as teeth. This cleaning helps prevent tooth decay and promote gingival health. In traditional dentifrice compositions abrasives aid in the removal of the pellicle film. This film usually comprises a thin acellular, glycoprotein-mucoprotein coating which adheres to the enamel within minutes after teeth are cleaned. The presence of various food pigments lodged within the film accounts for most instances of teeth discoloration.

Additionally, antiplaque agent(s) can be included in the dentifrice composition. Antiplaque agent(s) provide benefits beyond pellicle cleaning. The formation of dental plaque is the primary source of dental caries, gingival and periodontal disease, and tooth loss. Plaque is a mixed matrix of bacteria, epithelial cells, leukocytes, macrophages and other oral exudates. The bacteria associated with plaque can secrete enzymes and endotoxins which can irritate the gums and cause an inflammatory gingivitis. As the gums become increasingly irritated by this process, they have a tendency to bleed, lose their toughness and resiliency, and separate from the teeth. This separation results in periodontal pockets leading in turn to further accumulation of debris, secretions, and more bacteria/toxins. This process eventually leads to destruction of both the hard and soft tissue of the oral cavity.

The use of a variety of agents to clean the oral cavity and reduce plaque and mouth malodor is described in U.S. Pat. Nos. 3,696,191, 3,991,177, 4,058,595, 4,115,546, 4,138,476, 4,140,758, 4,154,815, 4,737,359, 4,986,981, 4,992,420, 5,000,939, 4,652,444, 4,725,428, 4,355,022, U.S. patent applications Ser. No. 02/105,898, 03/128,313, 03/223,209, and PCT application WO 86/02831.

In spite of the many disclosures relating to compositions for pellicle cleaning and antiplaque activity, the need for improved products still exists. The present invention has developed oral compositions which provide pellicle cleaning and antiplaque activity while minimizing the use of abrasives; thereby gently cleaning the surfaces of the oral cavity. Specifically, the present invention includes oral dentifrice compositions which deliver a chemical cleaning with a combination of chelant, surfactant and pH, while maintaining low levels or is substantially free of abrasives. In addition to gently cleaning the surfaces of the oral cavity, this composition can maximize the flavor and cleaning impact without increasing the levels of flavor and cleaning additives; as abrasives such as silica often absorb these additives.

### SUMMARY OF THE INVENTION

The present invention relates to a dentifrice composition comprising an oral care carrier, from about 0% to about 10% of an abrasive by weight of the total dentifrice composition, a fluoride, from about 2.5% of a chelating agent, from about 0.5% to about 5% of a surfactant, wherein the dentifrice composition has a pH greater than about 8, and wherein the dentifrice composition has a PCR of at least 20 and wherein greater than 10% of the PCR value results from chemical cleaning.

The present invention further relates to a dentifrice composition comprising an oral care carrier, from about 0% to about 5% of an abrasive by weight of the total dentifrice composition, a fluoride, from about 2.5% of a chelating agent, from about 0.5% to about 5% of a surfactant, wherein the dentifrice composition has a pH greater than about 8, and wherein the dentifrice composition has a PCR of at least 20 and wherein greater than 20% of the PCR value results from chemical cleaning.

The present invention further relates to a dentifrice composition comprising an oral care carrier, from about 0% to about 5% of an abrasive by weight of the total dentifrice composition, a fluoride, from about 0.5% to about 5% of a chelating surfactant, wherein the dentifrice composition has a pH greater than about 8, and wherein the dentifrice composition has a PCR of at least 20 and wherein greater than 20% of the PCR value results from chemical cleaning.

### DETAILED DESCRIPTION OF THE INVENTION

A. Definitions

The term “orally active” as used herein means a material that provides either a cosmetic, prophylactic or therapeutic benefit within the oral cavity.

The term “teeth”, as used herein, is meant to include natural teeth, dentures, dental plates, fillings, caps, crowns, bridges, dental implants, and the like, and any other hard surfaced dental prosthesis either permanently or temporarily fixed within the oral cavity.

By “safe and effective amount”, as used herein, is meant an amount of an agent (e.g., anti-calculus agent) high enough to significantly improve the condition to be treated, but low enough to avoid serious side effects (at a reasonable benefit/risk ratio), within the scope of sound medical/dental judgment. The safe and effective amount of an agent (e.g., anti-calculus agent) may vary with the particular condition being treated, the age and physical condition of the patient being treated, the severity of the condition, the duration of treatment, the nature of concurrent therapy, the specific form of the source employed, and the particular vehicle from which the agent is applied.

By “dentifrice” or “dentifrice composition” as used herein is meant paste, powder, tooth gel, and/or liquid formulations used to clean the surfaces of the oral cavity. The dentifrice is an oral composition that is not intentionally swallowed for purposes of systemic administration of therapeutic agents, but is retained in the oral cavity for a sufficient time to contact substantially all of the dental surfaces and/or mucosal tissues for purposes of oral activity. In addition dentifrice can mean a product which may be intentionally swallowed but not swallowed for the purposes of systemic administration of therapeutic agents.

By “oral condition” as used herein is meant diseases or conditions of the oral cavity including caries, plaque, breath malodor, dental erosion, gingivitis, and periodontal disease. Oral conditions are further described in WO 02/02096A2, published Jan. 10, 2002, P&G.

By “tooth surfaces” or “teeth surfaces” as used herein is meant the pits, fissures, occlusal surfaces, cleft, crevices, grooves, depressions, interstices, irregularities, inter-proximal surfaces between the teeth and/or along the gum line, the smooth surfaces of teeth, and/or the grinding or biting surfaces of a tooth.

By “whole body health” as used herein is meant overall systemic health characterized by a reduction in risk of development of major systemic diseases and conditions including cardiovascular disease, stroke, diabetes, severe respiratory infections, premature births and low birth weights (including post-partum dysfunction in neurologic/development function), and associated increased risk of mortality. It is believed that oral infections could lead to systemic infection. Bacteria can spread from the mouth into the bloodstream and other parts of the body, thereby putting a person's health at risk. Oral infection may contribute to the development of a number of serious conditions including heart disease, diabetes, respiratory diseases and premature, underweight births. Whole body health and promotion thereof by treating oral cavity infections is further described in WO 02/02063A2, WO 02/02096A2, WO 02/02128A2, all published Jan. 10, 2002.

All percentages and ratios used hereinafter are by weight of total dentifrice composition, unless otherwise indicated.

All measurements referred to herein are made at 25° C. unless otherwise specified.

All percentages, ratios, and levels of ingredients referred to herein are based on the actual amount of the ingredient, and do not include solvents, fillers, or other materials with which the ingredient may be combined as a commercially available product, unless otherwise indicated.

B. Low Abrasive Chemical Cleaning System

A significant portion of the cleaning delivered by the inventive dentifrice composition results from the action of chemicals (hereinafter “chemical cleaning”) rather than physical abrasion resulting from an abrasive component of the dentifrice composition (hereinafter “abrasive cleaning”). This chemical cleaning softens the plaque and biofilm deposits on the teeth making these deposits easier to remove with brushing. This results in the removal of deposits with brushing or with brushing and the addition of minimal levels of abrasive components such as silica. The chemical cleaning is delivered via a combination of chelant level, surfactant level, and pH and/or the use of chelating surfactant and pH. The dentifrice composition of the present invention delivers cleaning equivalent to or greater than dentifrice compositions having traditional levels of abrasives (in excess of 10% abrasive by weight of the total dentifrice composition), while maintaining a low level or a dentifrice composition substantially free of abrasives. Low level of abrasives, as used herein, is from about 5% to about 10% by weight of the total dentifrice composition. Substantially free of abrasives, as used herein, is from about 0 to about 5% by weight of the total dentifrice composition. The dentifrice composition comprising low level abrasive or the dentifrice composition substantially free of abrasive gently cleans the surfaces of the oral cavity while minimizing abrading or scratching of these oral surfaces. In addition to gently cleaning the oral surfaces, minimizing the level of abrasive in the dentifrice composition can reduce the amount of other components needed to deliver the desired benefit. For example, abrasives such as silica are known to generate compatibility problems with flavor components, fluoride and cetylpyridinium chloride (hereinafter “CPC”). Therefore, minimizing the level of abrasives in the dentifrice composition maximizes the impact of flavor and CPC, and improves the stability of fluoride.

The chemical cleaning provided by the present invention with only a minimal amount of abrasive can also provide protection against excessive tooth wear, including excessive tooth wear that can occur as a result of dental erosion. The chemical cleaning of the present invention will minimize any dental erosion. Dental erosion is a permanent loss of tooth substance from the surface by the action of harsh or unsafe abrasives and/or acids, such as acidic beverages and juices. The dentifrice composition of the present invention has a low level or is substantially free of an abrasive and still provides the necessary cleaning. This low level of abrasion can aid in the protection of teeth from excessive tooth wear and minimize any dental erosion.

The cleaning resulting from the use of the dentifrice composition can be measured with a pellicle cleaning ratio test, hererinafter “PCR.” The oral cleaning resulting from use of the inventive dentifrice composition is provided at least in part by the chemistry of the dentifrice composition. In particular the chemistry can aid in the removal of the pellicle film, and thus this film removal is not solely a result of the abrasive that may be included in the dentifrice composition. For example, in one embodiment from about 10% to about 100% of the PCR value results from chemical cleaning and less than about 90% of the PCR value results from abrasive cleaning. In another embodiment from about 15% to about 100% of the PCR value results from chemical cleaning and less than about 85% of the PCR value results from abrasive cleaning. In yet another embodiment from about 20% to about 100% of the PCR value results from chemical cleaning and less than about 80% of the PCR value results from abrasive cleaning. In yet another embodiment from about 25% to about 100% of the PCR value results from chemical cleaning and less than about 75% of the PCR value results from abrasive cleaning; and in yet another embodiment of the invention from about 50% to about 100% of the PCR value results from chemical cleaning and less than about 50% of the PCR value results from abrasive cleaning. In other words, from about 10% to about 75% of the cleaning is chemical cleaning, and from about 0 to about 75% of the cleaning is abrasive cleaning. In another embodiment from about 10% to about 50% of the cleaning is chemical cleaning, and in another embodiment from about 10% to about 25% of the cleaning is chemical cleaning, and in yet another embodiment from about 15% to about 50% of the cleaning is chemical cleaning, and in yet another embodiment from about 20% to about 50% of the cleaning is chemical cleaning.

The PCR (Pellicle Cleaning Ratio) cleaning values are determined by a slightly modified version of the PCR test described in “In Vitro Removal of Stain With Dentifrice”, G. K. Stookey, T. A. Burkhard and B. R. Schemerhorn, J. Dental Research, 61, 1236-9, 1982. Cleaning is assessed in vitro by use of the modified pellicle cleaning ratio test. This test is identical to that described by Stookey et al. with the following modifications: (1) a clear artificial pellicle film is applied to bovine chips prior to application of the stained film, (2) solution heating is used rather than radiative heating during film application, (3) the number of brush strokes is reduced to 1000 strokes and (4) the slurry concentration is 1 part dentifrice to 3 parts water. To determine the percentage of the PCR value attributable to chemical cleaning vs. the percentage of the PCR value attributable to abrasive cleaning, the PCR test is run with both the dentifrice composition in solution, and the dentifrice composition in solution with the abrasive removed.

As defmed herein, the term “abrasive” includes any component of the dentifrice composition which settles to the bottom of a centrifuge tube when the dentifrice composition is diluted 3:1 with water, agitated, placed in a centrifuge tube and spun at 10,000 rpm for fifteen minutes at room temperature ±5° C. The components that make up the “pellet” that settles to the bottom of the tube are the abrasives of the dentifrice composition. The PCR test is run with the dentifrice composition diluted 3:1 with water (hereinafter “diluted dentifrice composition”) and again with the supernatant (solution suspended above the pellet) that results from the centrifuging of the dentifrice composition diluted 3:1 with water (hereinafter “supernatant”). The percentage of the PCR attributable to the chemical cleaning is calculated using the following formula:
(PCR of supernatant/PCR of the diluted dentifrice composition)×100=% of PCR value resulting from chemical cleaning

The dentifrice composition of the present invention includes a surfactant (from about 0.5% to about 5%), a chelating agent (from about 2.5% to about 15%), and may include one or more of the following: a plaque or stain specific solvent (from about 0.001% to about 10%), a thickening agent (from about 0.1% to about 5%), a humectant (from about 10% to about 55%), a sweetening agent (from about 0.1% to about 3%), a flavor agent (from about 0.001% to about 10%), a coloring agent (from about 0.01% to about 0.5%), an abrasive (from about 0% to about 10%) and water (from about 2% to about 45%). Such dentifrice composition may also include one or more of an additional anticaries agent (from about 0.05% to about 10% additional anticaries agent), and an anticalculus agent (from about 0.1% to about 13%). The tooth powders, of course, contain substantially all non-liquid components.

Additional suitable carriers are disclosed in U.S. Pat. Nos. 5,198,220, and 5,242,910.

1. Chelant

A chelating agent can be selected from the group consisting of tartaric acid and pharmaceutically-acceptable salts thereof, citric acid and alkali metal citrates and mixtures thereof. Chelating agents are able to complex calcium found in the cell walls of the bacteria. Chelating agents can also disrupt plaque by removing calcium from the calcium bridges which help hold this biomass intact. However, it is possible to use a chelating agent which has an affinity for calcium that is too high. This results in tooth demineralization and is contrary to the objects and intentions of the present invention.

In one embodiment of the dentifrice composition the chelant alkali metal citrate can comprise potassium citrate or sodium citrate and/or mixtures thereof. In another embodiment the chelant is a citric acid/alkali metal citrate combination. In yet another embodiment the dentifrice composition comprises alkali metal salts of tartaric acid. In yet another embodiment the dentifrice composition comprises disodium tartrate, dipotassium tartrate, sodium potassium tartrate, sodium hydrogen tartrate and potassium hydrogen tartrate. In one embodiment the level of chelating agent suitable for use in the present invention is from about 2.5% to about 15% by weight of the total dentifrice composition and in yet another embodiment the level is from about 5% to about 10% by weight of the total dentifrice composition, and in yet another embodiment the chelating agent is from about 4% to about 7% by weight of the total dentifrice composition, and in yet another embodiment the chelating agent is from about 2.5, 3, 4, 5, 6, 7, 8, 9, 10, 11, 12, 13, 14% by weight of the total dentifrice composition up to about 15, 14, 13, 12, 11, 10, 9, 8, 7, 6, 5, 4, 3% and any combination thereof by weight of the total dentifrice composition. The tartaric acid salt chelating agent can be used alone or in combination with other chelating agents.

Other chelating agents can be used. These chelating agents can have a calcium binding constant of about 10¹ to 10⁵ provide improved cleaning with reduced plaque and calculus formation.

Another group of agents suitable for use as chelating agents in the present invention are the soluble pyrophosphates. The pyrophosphate salts used in the present compositions can be any of the alkali metal pyrophosphate salts and/or any combination thereof. Specific salts include tetra alkali metal pyrophosphate, dialkali metal diacid pyrophosphate, trialkali metal monoacid pyrophosphate and mixtures thereof, wherein the alkali metals can be sodium or potassium. The salts are useful in both their hydrated and unhydrated forms. In one embodiment an effective amount of pyrophosphate salt useful in the present composition can be enough to provide at least about 1.0% pyrophosphate ion, in another embodiment from about 1.5% to about 6%, in yet another embodiment from about 3.5% to about 6% of such ions. It is to be appreciated that the level of pyrophosphate ions is that capable of being provided to the composition (i.e., the theoretical amount at an appropriate pH) and that pyrophosphate forms other than P207-4 (e.g., (HP207-3)) may be present when a final product pH is established.

The pyrophosphate salts are described in more detail in Kirk & Othmer, Encyclopedia of Chemical Technology, Second Edition, Volume 15, Interscience Publishers (1968).

Still another possible group of chelating agents suitable for use in the present invention are the anionic polymeric polycarboxylates. Such materials are well known in the art, being employed in the form of their free acids or partially or fully neutralized water soluble alkali metal (e.g. potassium and sodium) or ammonium salts. In one embodiment the chelating agents are 1:4 to 4:1 copolymers of maleic anhydride or acid with another polymerizable ethylenically unsaturated monomer, including methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. Nos. 4,138,477, and 4,183,914, including copolymers of maleic anhydride with styrene, isobutylene or ethyl vinyl ether, poly-acrylic, polyitaconic and polymaleic acids, and sulfoacrylic oligomers of M.W. as low as 1,000 available as Uniroyal ND-2.

2. Surfactant

To deliver the desired cleaning, the dentifrice composition of the present invention has a surfactant level of from about 0.5% to about 5% by weight of the total dentifrice composition. In one embodiment the surfactant level is from about 1% to about 3% surfactant by weight of the total dentifrice composition. Suitable surfactant agents are those which are reasonably stable and form foam throughout a wide pH range. Surfactant agents include nonionic, anionic, amphoteric, cationic, zwitterionic, synthetic detergents, and mixtures thereof. Many suitable nonionic and amphoteric surfactants are disclosed in U.S. Pat. Nos. 3,988,433; and 4,051,234, and many suitable nonionic surfactants are disclosed in U.S. Pat. No. 3,959,458. In one embodiment the surfactant is sodium lauryl sulfate.

In one embodiment a surfactant having chelating properties is used. A suitable chelating surfactant, such as a surfactant comprising phosphonate groups, is disclosed in U.S. Patent Application Publication No. 2005/0153938, and includes 2,2-diphosphono-5-hydroxy-3-oxa-6-hexyltrimethylammonium chloride and 6-trimethylammoniuhexyl-1,1-bisphosphonic acid. The use of chelating surfactants in the dentifrice composition of the present invention can provide both the function of the surfactant and/or the function of the chelating agent. This chelating surfactant can be used at levels of from about 0.1 to about 5% by weight of the total composition and additionally can be included in the dentifrice composition in combination with the existing surfactant and/or chelating agent or can be used to replace all or a portion of the surfactant and/or chelating agent.

3. pH

In one embodiment the pH of the dentifrice compositions described herein ranges from about 6.5 to about 10, in another embodiment the pH is from about 8 to about 10 and in yet another embodiment the pH is about 8.

4. Fluoride Ion

The present invention comprises a safe and effective amount of a fluoride compound (e.g. water soluble). The fluoride ion is present in an amount sufficient to give a fluoride ion concentration in the composition at 25° C., and/or in one embodiment can be used at levels of from about 0.0025% to about 5.0% by weight, in another embodiment from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are disclosed in U.S. Pat. Nos. 3,535,421, and 3,678,154. Representative fluoride ion sources include: stannous fluoride, sodium fluoride, potassium fluoride, sodium monofluorophosphate and many others. In one embodiment the dentifrice composition comprises stannous fluoride or sodium fluoride, as well as mixtures thereof.

5. Optional Ingredients

a. Dentifrice Carrier

Carriers suitable for the preparation of dentifrice compositions of the present invention are well known in the art. Their selection will depend on secondary considerations like taste, cost, and shelf stability, etc. A dentifrice carrier can be used for a tooth paste, tooth gels, chewable tablets etc. such as disclosed in, U.S. Pat. No. 3,988,433 (e.g., abrasive materials, surfactants, binders, humectants, flavoring and sweetening agents, etc.).

b. Abrasive

A dentifrice composition of the present invention may comprise either low levels or be substantially free of abrasives. The material selected is to be compatible within the dentifrice composition of interest and does not excessively abrade dentin. If present, suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present dentifrice compositions is the particulate thermo-setting polymerized resins as described in U.S. Pat. No. 3,070,510. Suitable resins include, for example, melamines, phenolics, ureas, melamine-ureas, melamine-formaldehydes, urea-formaldehyde, melamine-urea-formaldehydes, cross-linked epoxides, and cross-linked polyesters. Mixtures of abrasives may also be used.

Silica dental abrasives of various types can be used because of their unique benefits of exceptional dental cleaning and polishing performance without unduly abrading tooth enamel or dentine. The silica abrasive polishing materials herein, as well as other abrasives, can have an average particle size ranging between about 0.1 to about 30 microns, and in another embodiment from about 5 to about 15 microns. The abrasive can be precipitated silica or silica gels such as the silica xerogels described in U.S. Pat. Nos. 3,538,230 and 3,862,307. Suitable silica xerogels are the silica xerogels marketed under the trade name “Syloid” by the W.R. Grace & Company, Davison Chemical Division. Also precipitated silica materials, such as those marketed by the J. M. Huber Corporation under the trade name, Zeodent®, can be used; in one embodiment the silica carrying the designation Zeodent 109® can be used. The types of silica dental abrasives useful in the toothpastes of the present invention are described in more detail in U.S. Pat. No. 4,340,583. Examples of suitable precipitated silica are the silica disclosed in U.S. Pat. Nos. 5,603,920; 5,589,160; 5,658,553; 5,651,958; and 6,740,311.

The abrasive in the dentifrice composition described herein is generally present at from about 0% to about 10% abrasive by weight of the dentifrice composition. In another embodiment the dentifrice composition comprises an abrasive of from about 0.01% to about 10% by weight of the total dentifrice composition. In yet another embodiment the dentifrice composition comprises an abrasive of from about 0.1% to about 10% by weight of the total composition. In another embodiment the dentifrice composition comprises from about 0% to about 5% of abrasive by weight of the dentifrice composition. In yet another embodiment the dentifrice composition comprises from about 5 to about 10% abrasive by weight of the dentifrice composition. In yet another embodiment the dentifrice composition comprises from about 2.5% to about 5% of abrasive by weight of the dentifrice composition, and in yet another embodiment the dentifrice composition comprises from about 1% to about 3% of abrasive by weight of the dentifrice composition. In yet another embodiment the dentifrice composition comprises an abrasive of greater than about 0.01, 0.1, 0.5, 1, 2, 2.5, 3, 4, 5, 6, 7, 8, 9% and less than about 10, 9, 8, 7, 6, 5, 4, 3, 2, 1% by weight of the total dentifrice composition.

c. Plaque or Stain Specific Solvent

The cleaning of the chemical cleaning system can be enhanced with solvents specific to the soils in the oral cavity. Examples of such solvents include, but are not limited to, limonene, PVP and alternate co-polymers, monoethanolamine, 1-pentanol and botanical extracts such as those disclosed in U.S. patent application Ser. No. 11/217,274 filed on Sep. 1, 2005.

d. Thickening Agents

In preparing toothpaste or gels, it is necessary to add some thickening material to provide a desirable consistency of the dentifrice composition, to provide desirable release characteristics upon use, to provide shelf stability, and to provide stability of the dentifrice composition, etc. Suitable thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or fmely divided silica can be used as part of the thickening agent to further improve texture.

Thickening agents can include however, except polymeric polyether compounds, e.g., polyethylene or polypropylene oxide (M.W. 300 to 1,000,000), capped with alkyl or acyl groups containing 1 to about 18 carbon atoms.

A suitable class of thickening or gelling agents includes a class of homopolymers of acrylic acid crosslinked with an alkyl ether of pentaerythritol or an alkyl ether of sucrose, or carbomers. Carbomers are commercially available from B.F. Goodrich as the Carbopol® series. Particularly the carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. Nos. 5,198,220; 5,242,910; and 4,443,430.

Thickening agents in an amount from about 0.1% to about 15%, or from about 0.2% to about 6%, in another embodiment from about 0.4% to about 5%, by weight of the total dentifrice composition, can be used.

e. Humectants

Another optional component of the topical, oral carriers of the dentifrice compositions of the present invention is a humectant. The humectant serves to keep dentifrice compositions from hardening upon exposure to air, to give dentifrice compositions a moist feel to the mouth, and, for particular humectants, to impart desirable sweetness of flavor to dentifrice compositions. The humectant, on a pure humectant basis, can comprise from about 0% to about 70%, and in another embodiment from about 5% to about 25%, by weight of the dentifrice compositions herein. Suitable humectants for use in dentifrice compositions of the subject invention include edible polyhydric alcohols such as glycerin, sorbitol, xylitol, butylene glycol, polyethylene glycol, and propylene glycol, especially sorbitol and glycerin.

f. Sweetening Agents

Sweetening agents which can be used include sucrose, glucose, saccharin, aspartame, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A dentifrice composition of the present invention can contain from about 0.1% to about 10% of these agents, in another embodiment from about 0.1% to about 1%, by weight of the dentifrice composition.

In addition to sweetening agents, coolants, salivating agents, warming agents, and numbing agents can be used as optional ingredients in dentifrice compositions of the present invention. These agents are present in the dentifrice compositions at a level of from about 0.001% to about 10%, in another embodiment from about 0.1% to about 1%, by weight of the dentifrice composition.

The coolant can be any of a wide variety of materials. Included among such materials are carboxamides, menthol, ketals, diols, and mixtures thereof. Suitable coolants in the present dentifrice compositions include the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425. WS-3 and other agents are described in U.S. Pat. No. 4,136,163.

Suitable salivating agents of the present invention include Jambu® manufactured by Takasago. Suitable warming agents include capsicum and nicotinate esters, such as benzyl nicotinate. Suitable numbing agents include benzocaine, lidocaine, clove bud oil, and ethanol.

g. Flavor Agents

Suitable flavoring agents or flavor compositions can also be added to the dentifrice composition. Suitable flavoring agents include, but are not limited to, oil of wintergreen, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, chocolate, green tea, and mixtures thereof. In on embodiment, the flavor composition may be selected from the group consisting of orange, lemon, cinnamon, spearmint, and combinations thereof. Coolants may also be part of the flavor composition. A flavor composition is generally used in the dentifrice compositions at levels of from about 0.001% to about 10%, by weight of the dentifrice composition. The flavor composition will preferably be present in an amount of from about 0.01% to about 5%, more preferably from about 0.1% to about 3%, and more preferably from about 0.5% to about 1.5% by weight. Other extracts from botanicals, particularly plants, can be added. Nonlimiting examples include grape, pomegranate, and cranberry. The extracts, flavor, coolants, salivating agents, anti-adhesion, anti-plaque, tongue coating agents, and other additive can be used to contribute to a clean or slick tooth feel or impression.

h. Cosmetic or Therapeutic Actives

The dentifrice composition may also comprise suitable cosmetic and/or therapeutic actives. Such actives include any material that is generally considered safe for use in the oral cavity and that provides changes to the overall appearance and/or health of the oral cavity, including, but not limited to, anti-calculus agents, fluoride ion sources, stannous ion sources, whitening agents, anti-microbial, anti-plaque agents, anti-inflammatory agents, nutrients, antioxidants, anti-viral agents, analgesic and anesthetic agents, H-2 antagonists, components which impart a clean feel to the teeth, pigments and colorants, fragrances and sensates, and mixture thereof. When present, the level of cosmetic and/or therapeutic active in the dentifrice composition is, in one embodiment from about 0.001% to about 90%, in another embodiment from about 0.01% to about 50%, and in another embodiment from about 0.1% to about 30%, by weight of the dentifrice composition.

The following is a non-limiting list of actives that may be used in the present invention.

1) Anticalculus Agent

Dentifrice compositions of the present invention may also comprise an anti-calculus agent, which in one embodiment may be present from about 0.05% to about 50%, by weight of the dentifrice composition, in another embodiment is from about 0.05% to about 25%, and in another embodiment is from about 0.1% to about 15%. The anti-calculus agent may be selected from the group consisting of polyphosphates (including pyrophosphates) and salts thereof; polyamino propane sulfonic acid (AMPS) and salts thereof; polyolefm sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof; polyolefin phosphonates and salts thereof; polypeptides; and mixtures thereof. In one embodiment, the salts are alkali metal salts. Polyphosphates are generally employed as their wholly or partially neutralized water-soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation, which are commercially known as Sodaphos (n≈6), Hexaphos (n≈13), and Glass H (n≈21, sodium hexametaphosphate), and mixtures thereof. The pyrophosphate salts useful in the present invention include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. Polyolefin sulfonates include those wherein the olefin group contains 2 or more carbon atoms, and salts thereof. Polyolefm phosphonates include those wherein the olefin group contains 2 or more carbon atoms. Polyvinylphosphonates include polyvinylphosphonic acid. Diphosphonates and salts thereof include azocycloalkane-2,2-diphosphonic acids and salts thereof, ions of azocycloalkane-2,2-diphosphonic acids and salts thereof, azacyclohexane-2,2-diphosphonic acid, azacyclopentane-2,2-diphosphonic acid, N-methyl-azacyclopentane-2,3-diphosphonic acid, EHDP (ethane-1-hydroxy-1,1,-diphosphonic acid), AHP (azacycloheptane-2,2-diphosphonic acid), ethane-1-amino-1,1-diphosphonate, dichloromethane-diphosphonate, etc. Phosphonoalkane carboxylic acid or their alkali metal salts include PPTA (phosphonopropane tricarboxylic acid), PBTA (phosphonobutane-1,2,4-tricarboxylic acid), each as acid or alkali metal salts. Polyolefin phosphates include those wherein the olefm group contains 2 or more carbon atoms. Polypeptides include polyaspartic and polyglutamic acids.

2) Stannous Ion

The dentifrice compositions of the present invention may include a stannous ion source. The stannous ions may be provided from stannous fluoride and/or other stannous salts. Stannous fluoride has been found to help in the reduction of gingivitis, plaque, sensitivity, and in improved breath benefits. The stannous ions provided in a dentifrice composition will provide efficacy to a subject using the dentifrice composition. Although efficacy could include benefits other than the reduction in gingivitis, efficacy is defined as a noticeable amount of reduction in in situ plaque metabolism. Formulations providing such efficacy typically include stannous levels provided by stannous fluoride and/or other stannous salts ranging from about 3,000 ppm to about 15,000 ppm stannous ions in the total dentifrice composition. The stannous ion is present in an amount of from about 4,000 ppm to about 12,000 ppm, in one embodiment from about 5,000 ppm to about 10,000 ppm. Other stannous salts include organic stannous carboxylates, such as stannous acetate, stannous gluconate, stannous oxalate, stannous malonate, stannous citrate, stannous ethylene glycoxide, stannous formate, stannous sulfate, stannous lactate, stannous tartrate, and the like. Other stannous ion sources include, stannous halides such as stannous chlorides, stannous bromide, stannous iodide and stannous chloride dihydride. In one embodiment the stannous ion source is stannous fluoride in another embodiment, stannous chloride dihydrate. The combined stannous salts may be present in an amount of from about 0.001% to about 11%, by weight of the dentifrice compositions. The stannous salts may, in one embodiment, be present in an amount of from about 0.01% to about 7%, in another embodiment from about 0.1% to about 5%, and in another embodiment from about 1.5% to about 3%, by weight of the dentifrice composition.

3) Whitening Agent

A whitening agent may be included as an active in the present dentifrice compositions. The actives suitable for whitening are selected from the group consisting of alkali metal and alkaline earth metal peroxides, metal chlorites, perborates inclusive of mono and tetrahydrates, perphoshates, percarbonates, peroxyacids, and persulfates, such as ammonium, potassium, sodium and lithium persulfates, and combinations thereof. Suitable peroxide compounds include hydrogen peroxide, urea peroxide, calcium peroxide, carbamide peroxide, magnesium peroxide, zinc peroxide, strontium peroxide and mixtures thereof. In one embodiment the peroxide compound is carbamide peroxide. Suitable metal chlorites include calcium chlorite, barium chlorite, magnesium chlorite, lithium chlorite, sodium chlorite, and potassium chlorite. Additional whitening actives may be hypochlorite and chlorine dioxide. In one embodiment the chlorite is sodium chlorite. In another embodiment the percarbonate is sodium percarbonate. In one embodiment the persulfates are oxones. The level of these substances is dependent on the available oxygen or chlorine, respectively, that the molecule is capable of providing to bleach the stain. In one embodiment the whitening agents may be present at levels from about 0.01% to about 40%, in another embodiment from about 0.1% to about 20%, in another embodiment form about 0.5% to about 10%, and in another embodiment from about 4% to about 7%, by weight of the dentifrice composition.

4) Anti-Microbial Agent

Anti-microbial agents may be included in the dentifrice compositions of the present invention. Such agents may include, but are not limited to: 5-chloro-2-(2,4-dichlorophenoxy)-phenol, commonly referred to as triclosan; 8-hydroxyquinoline and its salts; copper II compounds, including, but not limited to, copper(II) chloride, copper(II) sulfate, copper(II) acetate, copper(II) fluoride and copper(II) hydroxide; phthalic acid and its salts including, but not limited to those disclosed in U.S. Pat. No. 4,994,262, including magnesium monopotassium phthalate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; iodine; sulfonamides; bisbiguanides; phenolics; delmopinol, octapinol, and other piperidino derivatives; niacin preparations; zinc or stannous ion agents; nystatin; grapefruit extract; apple extract; thyme oil; thymol; antibiotics such as augmentin, amoxicillin, tetracycline, doxycycline, minocycline, metronidazole, neomycin, kanamycin, cetylpyridinium chloride, and clindamycin; analogs and salts of the above; methyl salicylate; hydrogen peroxide; metal salts of chlorite; and mixtures of all of the above. Anti-microbial components may be present from about 0.001% to about 20% by weight of the dentifrice composition. In another embodiment the antimicrobial agents generally comprise from about 0.1% to about 5% by weight of the dentifrice compositions of the present invention.

5) Anti-Plaque Agent

The dentifrice compositions of the present invention may include an anti-plaque agent such as stannous salts, copper salts, strontium salts, magnesium salts or a dimethicone copolyol. The dimethicone copolyol is selected from C12 to C20 alkyl dimethicone copolyols and mixtures thereof. In one embodiment the dimethicone copolyol is cetyl dimethicone copolyol marketed under the Trade Name Abil EM90. The dimethicone copolyol in one embodiment can be present in a level of from about 0.001% to about 25%, in another embodiment from about 0.01% to about 5%, and in another embodiment from about 0.1% to about 1.5% by weight of the dentifrice composition.

6) Anti-Inflammatory Agent

Anti-inflammatory agents can also be present in the dentifrice compositions of the present invention. Such agents may include, but are not limited to, non-steroidal anti-inflammatory (NSAID) agents oxicams, salicylates, propionic acids, acetic acids and fenamates. Such NSAIDs include but are not limited to ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone and acetaminophen. Use of NSAIDs such as ketorolac are claimed in U.S. Pat. No. 5,626,838. Disclosed therein are methods of preventing and/or treating primary and reoccurring squamous cell carcinoma of the oral cavity or oropharynx by topical administration to the oral cavity or oropharynx of an effective amount of an NSAID. Suitable steroidal anti-inflammatory agents include corticosteroids, such as fluccinolone, and hydrocortisone.

7) Nutrients

Nutrients may improve the condition of the oral cavity and can be included in the dentifrice compositions of the present invention. Nutrients include minerals, vitamins, oral nutritional supplements, enteral nutritional supplements, and mixtures thereof. Useful minerals include calcium, phosphorus, zinc, manganese, potassium and mixtures thereof. Vitamins can be included with minerals or used independently. Suitable vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Oral nutritional supplements include amino acids, lipotropics, fish oil, and mixtures thereof. Amino acids include, but are not limited to L-Tryptophan, L-Lysine, Methionine, Threonine, Levocarnitine or L-carnitine and mixtures thereof. Lipotropics include, but are not limited to, choline, inositol, betaine, linoleic acid, linolenic acid, and mixtures thereof. Fish oil contains large amounts of. Omega-3 (N-3) polyunsaturated fatty acids, eicosapentaenoic acid and docosahexaenoic acid. Enteral nutritional supplements include, but are not limited to, protein products, glucose polymers, corn oil, safflower oil, medium chain triglycerides. Minerals, vitamins, oral nutritional supplements and enteral nutritional supplements are described in more detail in Drug Facts and Comparisons (loose leaf drug information service), Wolters Kluer Company, St. Louis, Mo., ©1997, pps. 3-17 and 54-57.

8) Antioxidants

Antioxidants are generally recognized as useful in dentifrice compositions. Antioxidants are disclosed in texts such as Cadenas and Packer, The Handbook of Antioxidants, © 1996 by Marcel Dekker, Inc. Antioxidants useful in the present invention include, but are not limited to, Vitamin E, ascorbic acid, Uric acid, carotenoids, Vitamin A, flavonoids and polyphenols, herbal antioxidants, melatonin, aminoindoles, lipoic acids and mixtures thereof.

9) Analgesic and Anesthetic Agents

Anti-pain or desensitizing agents can also be present in the dentifrice compositions of the present invention. Analgesics are agents that relieve pain by acting centrally to elevate pain threshold without disturbing consciousness or altering other sensory modalities. Such agents may include, but are not limited to: strontium chloride; potassium nitrate; sodium fluoride; sodium nitrate; acetanilide; phenacetin; acertophan; thiorphan; spiradoline; aspirin; codeine; thebaine; levorphenol; hydromorphone; oxymorphone; phenazocine; fentanyl; buprenorphine; butaphanol; nalbuphine; pentazocine; natural herbs, such as gall nut; Asarum; Cubebin; Galanga; scutellaria; Liangmianzhen; and Baizhi. Anesthetic agents, or topical analgesics, such as acetaminophen, sodium salicylate, trolamine salicylate, lidocaine and benzocaine may also be present. These analgesic actives are described in detail in Kirk-Othmer, Encyclopedia of Chemical Technology, Fourth Edition, Volume 2, Wiley-Interscience Publishers (1992), pp. 729-737.

10) H-1 and H-2 Antagonists

The present invention may also optionally comprise selective H-1 and H-2 antagonists including compounds disclosed in U.S. Pat. No. 5,294,433.

11) Pigments and Colorants

Pigments may be added to the dentifrice compositions herein to more precisely indicate the locations at which the dentifrice composition has actually been in contact. Additionally, these substances may be suitable for modifying the color of the teeth to satisfy the consumer. These substances comprise particles that when applied on the tooth surface modify that surface in terms of absorption and, or reflection of light. Such particles provide an appearance benefit when a film containing such particles is applied over the surfaces of a tooth or teeth. Pigments, dyes, colorants and lakes may also be added to modify the appearance of the dentifrice compositions herein to render the product more acceptable to the consumer. Appropriate pigment levels are selected for the particular impact that is desirable to the consumer. For example, for teeth that are particularly dark or stained one would typically use pigments in sufficient amounts to lighten the teeth. On the other hand, where individual teeth or spots on the teeth are lighter than other teeth, pigments to darken the teeth may be useful. The levels of pigments and colorants may be in the range of about 0.001% to about 20%, in one embodiment from about 0.01% to about 15% and in another embodiment from about 0.1% to about 10% by total weight of the dentifrice composition.

Pigments and colorants include inorganic white pigments, inorganic colored pigments, pearling agents, filler powders and the like; see Japanese Published Patent Application Kokai No. 9 [1997]-100215. Specific examples are selected from the group consisting of talc, mica, magnesium carbonate, calcium carbonate, magnesium silicate, aluminum magnesium silicate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder, methacrylate powder, polystyrene powder, silk powder, crystalline cellulose, starch, titanated mica, iron oxide titanated mica, bismuth oxychloride, and mixtures thereof. In one embodiment the pigments and colorants are those selected from the group consisting of titanium dioxide, bismuth oxychloride, zinc oxide, Opatint D&C Red 27, CI 16185:1 Acid 27 Lake E123, CI14720:1 Carmosoisine Aluminum Lake E122, Red 7 Lake, or Red 30 Lake and mixtures thereof.

12) Additional Actives

Additional actives suitable for use in the present invention may include, but are not limited to, insulin, steroids, herbal and other plant derived remedies. Additionally, anti-gingivitis or gum care agents known in the art may also be included. Components which impart a clean feel to the teeth may optionally be included. These components may include, for example, baking soda or Glass-H. Also, it is recognized that in certain forms of therapy, combinations of these above-named agents may be useful in order to obtain an optimal effect. Thus, for example, an anti-microbial and an anti-inflammatory agent may be combined in a single dentifrice composition to provide combined effectiveness.

Optional agents to be used include such known materials as synthetic anionic polymers, including polyacrylates and copolymers of maleic anhydride or acid and methyl vinyl ether (e.g., Gantrez), as described, for example, in U.S. Pat. No. 4,627,977, as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Additionally, the dentifrice composition can include a polymer carrier, such as those described in U.S. Pat. Nos. 6,682,722 and 6,589,512 and U.S. application Ser. Nos. 10/424,640 and 10/430,617.

13) Alkali Metal Bicarbonate Salt

The present invention may also include an alkali metal bicarbonate salt. Alkali metal bicarbonate salts are soluble in water and unless stabilized, tend to release carbon dioxide in an aqueous system. In one embodiment sodium bicarbonate, also known as baking soda, is the alkali metal bicarbonate salt. The present dentifrice composition may contain from about 0.5% to about 30%, in another embodiment from about 0.5% to about 15%, and in another embodiment from about 0.5% to about 5% of an alkali metal bicarbonate salt.

i. Miscellaneous Carriers

Water employed in the preparation of commercially suitable dentifrice compositions should be of low ion content and free of organic impurities. Water generally comprises from about 5% to about 70%, and in another embodiment from about 20% to about 50%, by weight of the dentifrice composition herein. These amounts of water include the free water which is added plus that which is introduced with other materials, such as with sorbitol.

Titanium dioxide may also be added to the present dentifrice composition. Titanium dioxide is a white powder which adds opacity to the dentifrice compositions. Titanium dioxide generally comprises from about 0.25% to about 5% by weight of the dentifrice compositions. The titanium dioxide may be mica coated.

Other optional agents include synthetic anionic polymeric polycarboxylates being employed in the form of their free acids or partially or fully neutralized water soluble alkali metal (e.g. potassium and sodium) or ammonium salts and are disclosed in U.S. Pat. Nos. 4,152,420, 3,956,480, 4,138,477, 4,183,914, and 4,906,456. In one embodiment the copolymers can be 1:4 to 4:1 copolymers of maleic anhydride or acid with another polymerizable ethylenically unsaturated monomer, in particular the methyl vinyl ether (methoxyethylene) can have a molecular weight (M.W.) of about 30,000 to about 1,000,000. These copolymers are available for example as Gantrez (AN 139 (M.W. 500,000), A.N. 119 (M.W. 250,000) and as S-97 Pharmaceutical Grade (M.W. 70,000), of GAF Corporation.

C. Methods of Use

A safe and effective amount of the dentifrice compositions of the present invention may be topically applied to the mucosal tissue of the oral cavity, to the gingival tissue of the oral cavity, and/or to the surface of the teeth, for the treatment or prevention of the above mentioned conditions of the oral cavity, in several conventional ways. For example the gingival/mucosal tissue and/or teeth are bathed in the liquid and/or lather generated by brushing the teeth with a dentifrice composition. A chewable dentifrice tablet can be chewed thus delivering the tooth actives to the surfaces of the oral cavity such as described in U.S. patent application Publication Nos. 2004/0101493 and 2004/0101494. The teeth can then be brushed after the tablet is chewed. Other non-limiting examples include applying the gel or paste, directly to the gingival/mucosal tissue or to the teeth with or without an oral care appliance described below.

For the method of treating diseases or conditions of the oral cavity, including caries, a safe and effective amount of the present dentifrice compositions can be applied to the gingival/mucosal tissue and/or the teeth of a person in need thereof, for at least about 10 seconds, in another embodiment from about 20 seconds to about 10 minutes, in even another embodiment from about 30 seconds to about 60 seconds. The method often involves expectoration of most of the dentifrice composition following such contact. The frequency of such contact can be from about once per week to about four times per day, in another embodiment from about thrice per week to about three times per day, and in another embodiment from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment.

D. Test Methods

Pellicle Cleaning Ratio

The PCR (Pellicle Cleaning Ratio) cleaning values are determined by a slightly modified version of the PCR test described in “In Vitro Removal of Stain With Dentifrice”, G. K. Stookey, T. A. Burkhard and B. R. Schemerhorn, J. Dental Research, 61, 1236-9, 1982. Cleaning is assessed in vitro by use of the modified pellicle cleaning ratio test. This test is identical to that described by Stookey et al. with the following modifications: (1) a clear artificial pellicle film is applied to bovine chips prior to application of the stained film, (2) solution heating is used rather than radiative heating during film application, (3) the number of brush strokes is reduced to 1000 strokes and (4) the slurry concentration is 1 part dentifrice to 3 parts water.

E. Examples

The dentifrice compositions of this invention are useful for both human and other lower animal (e.g. pets, zoo, or domestic animals) applications.

The following non-limiting examples further describe embodiments within the scope of the present invention. Many variations of these examples are possible without departing from the scope of the invention.

A dentifrice composition of the present invention contains the following components as described below.

| | Example Number | | | | |
|---|---|---|---|---|---|
| | 1 | 2 | 3 | 4 | 5 |
| Ingredient | Wt % | Wt % | Wt % | Wt % | Wt % |
| Sodium Fluoride, USP | 0.243 | 0.243 | 0.243 | 0.243 | 0.243 |
| Sodium | 1.5 | 1.5 | | 1.5 | 6.0 |
| Tripolyphosphate | | | | | |
| Saccharin Sodium USP | 0.48 | 0.48 | 0.48 | 0.48 | 0.48 |
| Granular | | | | | |
| Sodium Acid | 0.5 | 2.0 | 1.5 | 0.5 | 0.05 |
| Pyrophosphate | | | | | |
| Tetrasodium | 0.5 | 2.4 | 3.5 | 0.5 | |
| Pyrophosphate | | | | | |
| Xanthan Gum | 0.5 | 0.5 | 0.5 | 0.5 | 0.5 |
| Carbopol 956 | 1.2 | 1.2 | 1.2 | 1.2 | 1.2 |
| Glycerin | 29.0 | | | | 29.0 |
| Sorbitol Solution (70%) | 34.0 | 34.0 | 34.0 | 34.0 | 34.0 |
| USP Water | QS | QS | QS | QS | QS |
| Precipitated Silica, | 2.0 | 5.0 | 10.0 | 2.0 | 2.0 |
| Zeodent 109 | | | | | |
| FD&C Blue #1 | | 0.3 | 0.3 | 0.3 | 0.3 |
| (1% Soln.) | | | | | |
| Flavor | 2.15 | 2.35 | 1.75 | 2.36 | 2.15 |
| Sodium Lauryl Sulfate | 7.0 | 6.0 | 5.0 | | 7.0 |
| Solution (27.9%) | | | | | |
| Phosphonate Surfactant | | | | 4.0 | |
| Sodium hydroxide | 0.8 | 0.8 | 0.8 | 0.8 | 0.8 |
| (50% soln.) | | | | | |
| | 100 | 100 | 100 | 100 | 100 |

The humectants and thickening agents are added to the mixing tank and agitation is started. When the thickening agents are homogeneously dispersed into the humectant, water is added to the mix tank. Next fluoride, sweetening agents, buffering agents, chelant, coloring agents and titanium dioxide are added. If used, the abrasive is then added to the mixing tank with high agitation and vacuum. The surfactant and flavoring agents are added to the combination and mixing is continued. Mixing is continued for approximately 5 minutes. The resulting composition can have a pH of about 8.

The dimensions and values disclosed herein are not to be understood as being strictly limited to the exact numerical values recited. Instead, unless otherwise specified, each dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice comprising:
a. an oral care carrier;
b. a safe and effective amount of a fluoride ion source;
c. at least about 2.5% of a chelating agent;
d. from about 0.5% to about 5% of a surfactant;
wherein the dentifrice has a pH greater than about 8, and wherein the dentifrice has a PCR value of at least about 20 and wherein greater than about 10% of the PCR value results from chemical cleaning.

2. The dentifrice of claim 1, wherein the oral care carrier is a flavor having a range from about 0.5 to about 1.5% by weight of the total dentifrice.

3. The dentifrice of claim 1, further comprising an abrasive of from about 0.01% to about 10%.

4. The dentifrice of claim 1, wherein the pH is from about 8 to about 10.

5. The dentifrice of claim 1, wherein the surfactant is sodium lauryl sulfate.

6. The dentifrice of claim 1, wherein the dentifrice has a slick or clean tooth feel.

7. The dentifrice of claim 1, wherein from about 15% to about 100% of the PCR value results from chemical cleaning.

8. The dentifrice of claim 7, wherein from about 20% to about 100% of the PCR value results from chemical cleaning.

9. The dentifrice of claim 8, wherein from about 50% to about 100% of the PCR value results from chemical cleaning.

10. The dentifrice of claim 1, wherein the dentifrice has a PCR value of at least about 40.

11. A method of minimizing dental erosion of a subject's teeth, said method comprising administering to the subject's oral cavity the dentifrice composition of claim 1

12. A dentifrice comprising:
a. an oral care carrier;
b. a safe and effective amount of a fluoride ion source;
c. from about 2.5% to about 15% of a chelating agent;
c. from about 0.5% to about 5% of a surfactant;
wherein the dentifrice has a pH greater than about 8, and wherein the dentifrice has a PCR value of at least 20 and wherein greater than 20% of the PCR value results from chemical cleaning.

13. The dentifrice of claim 12, wherein the oral care carrier is a flavor having a range from about 0.5 to about 1.5% by weight of the total dentifrice.

14. The dentifrice of claim 12, further comprising an abrasive of from about 0.01% to about 10%.

15. The dentifrice of claim 12, wherein the pH is from about 8 to about 10.

16. The dentifrice of claim 12, wherein the surfactant is sodium lauryl sulfate.

17. The dentifrice of claim 12, wherein from about 20% to about 100% of the PCR value results from chemical cleaning.

18. The dentifrice of claim 12, further comprising a polyphosphate.

19. A method of improving whole body health by brushing the teeth with the dentifrice of claim 12.

20. A dentifrice comprising:
a. an oral care carrier;
b. a safe and effective amount of a fluoride ion source;
c. from about 0.5% to about 5% of a chelating surfactant;
wherein the dentifrice has a pH greater than about 8, and wherein the dentifrice has a PCR of at least 20 and wherein greater than 20% of the PCR value results from chemical cleaning.
